SEQUENCE LISTING (8 sequences)

SEQ ID NO: 1 - protein, 35 aa, Homo sapiens
HSGFEDELSEVLENQSSQAELKEAVEEPSSKDVME

SEQ ID NO: 2 - protein, 9 aa, synthetic construct
  REGION 1..9: note = Peptide Sequence
EDELSEVLE

SEQ ID NO: 3 - protein, 7 aa, synthetic construct
  REGION 1..7: note = Peptide Sequence
KEAVEEP

SEQ ID NO: 4 - protein, 7 aa, synthetic construct
  REGION 1..7: note = Peptide Sequence
SSKDVME

SEQ ID NO: 5 - protein, 8 aa, synthetic construct
  REGION 1..8: note = Peptide Sequence
HSGFEDEL

SEQ ID NO: 6 - protein, 8 aa, synthetic construct
  REGION 1..8: note = Peptide Sequence
PSSKDVME

SEQ ID NO: 7 - protein, 15 aa, synthetic construct
  REGION 1..15: note = Peptide Sequence
NQSSQAELKEAVEEP

SEQ ID NO: 8 - protein, 14 aa, synthetic construct
  REGION 1..14: note = Peptide Sequence
KEAVEEPSSKDVME

(19) United States
(12) Patent Application Publication
(10) Pub. No.: US 2025/0235502 A1
(43) Pub. Date: Jul. 24, 2025

(54) CARDIO-PROTECTIVE EFFECT OF VASOCONSTRICTION-INHIBITING FACTOR (VIF)

(71) Applicants: Joachim JANKOWSKI, Roetgen (DE); Vera JANKOWSKI, Roetgen (DE); Adelina BALEANU-CURAJ, Aachen (DE)

(72) Inventors: Elisa-Anamaria LIEHN, Stolberg (DE); Joachim JANKOWSKI, Roetgen (DE); Vera JANKOWSKI, Roetgen (DE); Adelina BALEANU-CURAJ, Aachen (DE)

(21) Appl. No.: 19/038,979

(22) Filed: Jan. 28, 2025

Related U.S. Application Data
(62) Division of application No. 17/428,508, filed on Aug. 4, 2021, now abandoned.

(30) Foreign Application Priority Data
Feb. 5, 2019 (DE) 10 2019 102 786.1

(51) Int. Cl. A61K 38/17; A61K 45/06; A61P 9/10

(52) U.S. Cl. CPC A61K 38/1709; A61K 45/06; A61P 9/10

(57) ABSTRACT

The present invention relates to a vasoconstriction-inhibiting factor (VIF) or a nucleic acid encoding it for the prevention and/or treatment of consequences of a heart disease. Furthermore, the present invention relates to pharmaceutical compositions containing the VIF and targeted (combination) therapies, in particular using the pharmaceutical compositions according to the invention. The present invention further relates to a kit for non-therapeutic in-vitro use containing the VIF or a nucleic acid encoding it.

### Technical field

The invention resides in the field of cardiovascular drugs, in particular relating to those therapeutics, which can be used specifically in the prevention and/or therapy of consequences of a heart disease, in particular a myocardial infarction.

### BACKGROUND OF THE INVENTION

High blood pressure and its consequences are one of the most common causes of death worldwide. High blood pressure often remains undetected as a silent threat until it manifests itself in critical secondary diseases and/or secondary damages. High blood pressure is a particular burden on the cardiovascular system. On the one hand, high blood pressure puts a particular strain on the heart itself, especially the left ventricle, which accomplishes/has to accomplish the high pressure and thus has to do extra work permanently. To continuously ensure this, the thick muscle layer of the heart (myocardium) is further enlarged. With increasing thickness, however, the oxygen supply to the inner muscle layers becomes increasingly difficult. Over a longer period of time, this can eventually lead to cardiac insufficiency, in which the heart is no longer able to supply the body with a sufficient amount of oxygen-rich blood. The high pressure in the blood vessels, on the other hand, causes the blood vessels to wear or harden, thus enabling the development of arteriosclerosis, in which in particular cholesterol esters and other fats are deposited in the vessel wall. These deposits constrict the blood vessels, which in turn can lead to a further rise in blood pressure due to the increased vascular resistance accompanied therewith. Especially in the long term, this drastically increases the risk of coronary heart disease, angina pectoris, myocardial infarction and stroke.

Since Nov. 13, 2017, the American College of Cardiology (ACC) and the American Heart Association (AHA) have described the following classification of blood pressure values (“New ACC/AHA High Blood Pressure Guidelines Lower Definition of Hypertension”):

| | Systolic | | Diastolic |
|---|---|---|---|
| | [mmHg] | | [mmHg] |
| Normal | <120 | and | <80 |
| Increased | <130 | and | <80 |
| Stage 1 | 130-139 | or | 80-89 |
| Stage 2 | ≥140 | or | ≥90 |
| Hypertensive | ≥180 | or | ≥120 |
| crisis | | | |

The European Society of Cardiology (ESC) and the European Society of Hypertension (ESH) continue to refer to a classification (Tran et Giang, “Changes in blood pressure classification, blood pressure goals and pharmacological treatment of essential hypertension in medical guidelines from 2003 to 2013”, IJC Metabolic & Endocrine 2 (2014), 1-10) published already in 2003:

| | Systolic | Diastolic |
|---|---|---|
| | blood pressure | blood pressure |
| | value [mmHg] | value [mmHg] |
| Optimal | <120 | <80 |
| Normal | 120-129 | 80-84 |
| Increased | 130-139 | 85-89 |
| Stage 1 | 140-159 | 90-99 |
| Stage 2 | 160-170 | 100-109 |
| Stage 3 | ≥180 | ≥110 |
| Isolated systolic | ≥140 | <90 |
| hypertension | | |

The renin-angiotensin system (or the entire renin-angiotensin-aldosterone system) and especially the underlying angiotensin peptides are essential for the regulation of blood pressure.

The enzyme renin is responsible for the activation of a cascade in which renin converts the previously inactive angiotensinogen into angiotensin I by its protease function. Angiotensin I is finally converted by the Angiotensin Converting Enzyme (ACE) into angiotensin II, which has a strong vasoconstrictive effect and promotes the release of other substances, e.g. the hormone vasopressin, which in turn have a blood pressure-increasing effect.

Salem et al., Identification of the Vasoconstriction-Inhibiting Factor (VIF), “A Potent Endogenous Cofactor of Angiotensin II Acting on the Angiotensin II Type 2 Receptor”, Circulation, 2015 describes a new peptide, Vasoconstriction Inhibiting Factor (VIF), which can interfere with the vasoconstrictive properties of angiotensin II by having an effect on the angiotensin II type 2 receptor. The VIF peptide as such and its formation in the human body is generally described. However, possible effects on human pathophysiology, especially with regard to cardiovascular diseases, and thus therapeutic applicability and utilization of the peptide are not studied.

In addition, it was found that in patients with heart failure the plasma concentration of VIF, among others, was increased (FIG. 1). It was unclear, however, whether and how the occurring phenomenon could be technically exploited. Furthermore, Salem et al. exclusively described the effect of VIF on vasoconstriction. Specific effects, or the targeted use of VIF for specific diseases, are not disclosed.

The problem of the present invention is to reveal specifically the mechanisms of action and the molecular mechanisms of VIF, especially in the context of cardiac events such as angina pectoris and myocardial infarction, in order to unlock its therapeutic potential. Special focus is placed on targeted studies to exploit the VIF in potential therapies of patients with cardiac diseases. In particular, further properties and VIF mutants should be investigated by targeted technical modifications of the VIF and its smaller peptides. The aim of the reveal and investigation is to provide a preparation or combination preparation that can be used in the treatment and prevention of cardiac diseases.

Previous treatment of heart diseases, especially myocardial infarction, often involves a chronic lowering of blood pressure and thus lifelong drug therapy. Examples of this are therapy with beta blockers, statins, ACE inhibitors or peptides such as serelaxin. However, the Phase III study RELAX-AHF-2 could not demonstrate a clinical benefit of the corresponding drug RLX030 (serelaxin). Possible side effects or long-term secondary damages due to all the established therapies cannot be excluded. Based on this, the primary problem of the present invention was to reveal new, improved forms of therapy for patients with heart diseases to facilitate therapy, for which VIF has not been described so far.

### Definitions

The terms “amino acid molecule/amino acid sequence”, “protein”, “peptide” or “polypeptide” are used interchangeably herein without reference to the length of a specific amino acid sequence. The term “amino acid” or “amino acid sequence” or “amino acid molecule” comprises any natural or chemically synthesized protein, peptide or polypeptide or modified protein, peptide, polypeptide and enzyme (polypeptide having a catalytic activity), the term “modified” comprising any recombinant, chemical or enzymatic modification of the protein, peptide, polypeptide and enzyme or the nucleic acid sequence encoding them.

The terms “sequence(s)” and “molecule(s)” are used interchangeably herein when referring to nucleic acid sequences/molecules or amino acid sequences/molecules.

The term “pharmaceutically acceptable” herein refers to those ingredients, materials, compositions and/or dosage forms that, within the scope of a medical consideration or within the definition of any medical regulatory and/or approval authority, are suitable for contact with the cells, tissues or components of a subject, i.e. humans and animals, including contact with malignant cells or tissues of a subject, without undue toxicity, irritation, allergic reaction or other complications or side effects consistent with an appropriate risk-benefit ratio for a subject/patient. In accordance with a preferred embodiment, one or more excipients are used as described below.

The term “subject”, as used herein, refers to a human or non-human animal. The term includes, but is not limited to, mammals (e.g., humans, other primates, pigs, rodents (e.g., mice, rats or hamsters), rabbits, guinea pigs, cows, horses, cats, dogs, sheep and goats). In one embodiment, the subject is a human being.

The term “heart disease” comprises not only classical diseases such as coronary heart disease, but also preferably pathological conditions and events of the heart and thus in particular myocardial infarction, Angina pectoris and ischemia in the heart tissue, among others.

The term “consequences of heart disease”, as used herein, does not include the occurrence of the disease itself, e.g. the occurrence of a myocardial infarction, but rather the functional and/or pathological phenomena associated therewith, e.g. a reduction in cardiac output or the area affected by the ischemia of the infarct.

The term “heart tissue” includes, but is not limited to, the pericardium, epicardium, pericardial sac, the fatty layer under the heart (Tela subepicardiaca), the myocardium with the heart muscle cells and the endocardium, as well as the arterial and venous vascular accesses to the heart tissue, especially the coronary vessels.

The term “infarction” describes a loss of tissue-especially through necrosis—as a consequence of an oxygen shortage (hypoxia), preferably due to insufficient blood flow (ischemia).

The terms “treat”, “treating”, “treatment” and “therapy”, as used herein, describe treatment in a mammal, e.g. in a human, including (a) preventing the consequences of a disease, i.e. halting its development; (b) alleviating the consequences of a disease, i.e. causing a decline in the functions or tissue worsened by the disease; and/or (c) curing the consequences of the disease. The terms “treatment” and “therapy” are used interchangeably and include any form of preventive and/or curative treatment or therapy.

The terms “prevent”, “prophylactic” or “prevention” mean that a prophylactic treatment has taken place before the onset of the disease or before the occurrence of the symptoms associated with a disease to be prevented. However, prevention does not always, and not necessarily, lead to the complete absence of the disease and its symptoms; thus, a mitigation or delay of the disease or its symptoms is also embraced by prevention as described herein.

The term “partial sequence”, as used herein in the context of nucleic acid sequences, amino acid sequences and/or peptide sequences, refers to a coherent/contiguous fragment which can be derived from a matrix sequence according to the present application. Therefore, a partial sequence usually comprises 3, 4, 5, 6, 7, 8, 9, 10 or more contiguous positions according to the matrix sequence, optionally including additional modifications.

Where reference is made in this application to a percentage of homology or identity of nucleic acid sequences or amino acid sequences, such values define those obtained by using the EMBOSS Water Pairwise Sequence Alignment (nucleotides) program(http://www.ebi.ac.uk/Tools/psa/emboss_water/nucleotide.html) for nucleic acids or the EMBOSS Water Pairwise Sequence Alignment (protein) program(http://www.ebi.ac.uk/Tools/psa/emboss_water/) for amino acids. These programs, provided by the European Molecular Biology Laboratory (EMBL) European Bioinformatics Institute (EBI) for local sequence alignments, use a modified Smith-Waterman algorithm (see http://www.ebi.ac.uk/Tools/psa/and Smith, T. F. & Waterman, M. S. “Identification of common molecular subsequences” Journal of Molecular Biology, 1981 147 (1):195-197). When performing an alignment, the standard parameters defined by EMBL-EBI are used. These parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

The pharmaceutical composition, as described herein, can be applied systemically or locally if relevant. In a systemic application, the pharmaceutical composition, or its active ingredients, is transferred into the blood system and/or lymphatic system via direct (e.g. intravenous injection) or indirect (e.g. orally via the gastrointestinal tract) routes, which allows for distribution throughout the body or in areas not separated by a specific barrier (e.g. blood-brain barrier). In a local application, the pharmaceutical composition is applied to the tissue in which it is intended to act. For example, a topical application or an injection can be used. In some embodiments, local application can also be made into adjacent tissue.

In one embodiment, the pharmaceutical composition is provided in an orally administrable form. The known pharmaceutical forms for such an application are particularly preferred, e.g. tablets (non-coated as well as coated tablets, e.g. with enteric coating), capsules, dragees, sprays, gels, bars, sachets, granules, pellets, syrups, solid mixtures, dispersions in liquid phases, emulsions, solutions, pastes or other swallowable or chewable pharmaceutical preparations and aqueous or oily suspensions. An orally administrable form is particularly, but not exclusively, advantageous for preventive therapy, as it ensures high patient compliance.

In another embodiment, the pharmaceutical composition may be available in an intravenously administrable form, e.g. as a solution. If applicable, administrable forms can be obtained from a mixture of the active ingredient and excipients. Such excipients may include fillers (such as sugar, sugar alcohols and cyclodextrins, thus e.g. sucrose, lactose, fructose, maltose, raffinose, sorbitol, lactitol, mannitol, maltitol, erythritol, inositol, trehalose, isomalt, inulin, maltodextrin, β-cyclodextrin, hydroxypropyl-β-cyclodextrin, sulfobutyl ether cyclodextrin or combinations thereof; calcium phosphate); carriers (such as polyethylene glycol (PEG), polyethylene oxide (PEO), polyvinylpyrrolidone (PVP), polyvinyl alcohol (PVA), hydroxypropylmethylcellulose (HMPC), hydroxypropylcellulose (HPC), carboxymethylethylcellulose (CMEC), hydroxypropylmethylcellulose phthalate (HPMCP), polyacrylate, polymethylacrylate, urea and sugar (e.g. mannitol)); polymers (such as polyvinylpyrrolidone, vinylpyrrolidone/vinyl acetate copolymer, polyalkylene glycol (e.g. polyethylene glycol), hydroxyalkyl cellulose (e.g. hydroxypropyl cellulose), hydroxyalkyl methyl cellulose (e.g. hydroxypropyl methyl cellulose), carboxymethyl cellulose, sodium carboxymethyl cellulose, ethyl cellulose, polymethacrylates (e.g. Eudragit® types), polyvinyl alcohol, polyvinyl acetate, vinyl alcohol/vinyl acetate copolymer, polyglycosylated glycerides, xanthan gum, carrageenan, chitosan, chitin, polydextrin, dextrins, starch and starch derivatives, proteins and their combinations); surfactants (such as sodium dodecyl sulfate, Brij 96, Tween 80); disintegrants (such as starch, e.g. sodium starch glycolate, corn starch or its derivatives); binders (such as povidone, crosspovidone, polyvinyl alcohols, hydroxypropyl methyl cellulose, microcrystalline cellulose, polyvinyl pyrrolidone); lubricants (such as stearic acid or its salts such as magnesium stearate, silicon dioxide, talc); sweeteners (such as aspartame); flavorings (such as β carotene); plasticizers (such as triethyl citrate, dibutyl phthalate); coating material (such as polyvinyl acetate phthalate, hydroxypropyl methyl cellulose phthalate); cooling agents (e.g. menthol derivatives (e.g. L-mentyllactate, L-menthyl alkyl carbonate, menthone ketals); recrystallization inhibitors; fluxes; defoamers; antioxidants; adsorbents; dyes; pH-modifying substances.

Likewise, a pharmaceutical composition according to the invention may contain preservatives, solvents, stabilizers, wetting agents, emulsifiers, salts for adjusting osmotic pressure, buffers or other components and substances customary for pharmaceutical compositions.

### BRIEF DESCRIPTION OF THE FIGURES

FIG. 1 shows the increased plasma concentration of VIF in patients with heart failure (NYHA classes III and IV) compared to controls (NYHA level<II). The NYHA classification is a scheme originally published by the New York Heart Association for classifying heart diseases according to severity. It is most commonly used to classify heart failure into different stages according to the patient's ability to perform. NYHA I: Heart disease without physical limitation. Everyday physical stress does not cause inadequate exhaustion, dysrhythmia, shortness of breath or angina pectoris. NYHA II: Heart disease with slight limitation of physical performance. No complaints at rest. Everyday physical stress causes exhaustion, rhythm disturbances, shortness of breath or angina pectoris. NYHA III: Heart disease with severe limitation of physical performance during usual activities. No complaints at rest. Low physical stress causes exhaustion, rhythm disturbances, shortness of breath or angina pectoris. NYHA IV: Heart disease with symptoms during all physical activities and at rest. Bedriddenness.

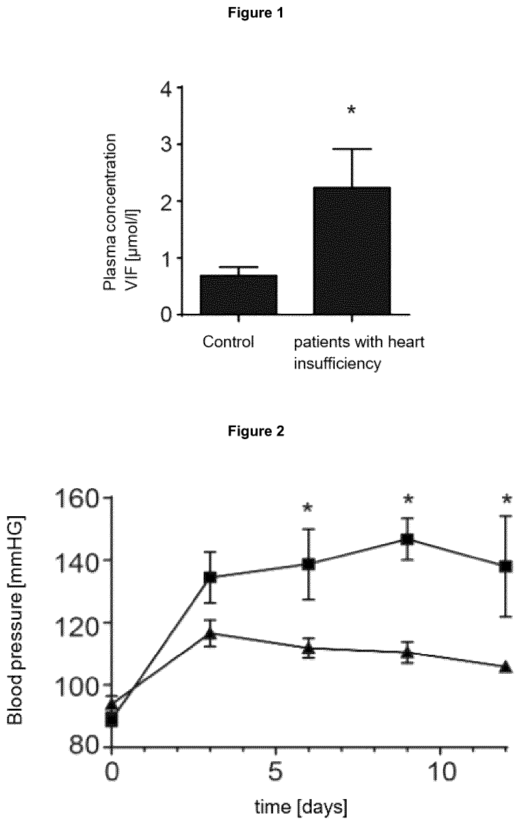

FIG. 2 shows the antihypertensive effect of VIF on male Wistar rats after subcutaneous application of angiotensin II (0.4 mg per kg per day) with (▴) or without (▪) intraperitoneal application of VIF (1 mg per ml).

FIG. 3 shows the size of the area affected by an induced myocardial infarction (as a percentage of the ventricle) and the ejection fraction after the induced myocardial infarction (as a percentage of the ejection fraction before myocardial infarction) in mice with preceding and following 2-day treatment with VIF each and mice without treatment with VIF.

FIG. 4 shows the size of the area affected by an induced myocardial infarction in the form of histological sections.

FIG. 5 shows (A) the influence of VIF treatment (A) on the ejection fraction of the heart after myocardial infarction and (B) the influence of VIF on blood pressure. CTR=control; preOP=control measurement before surgery in an animal model, as explained in more detail in the examples, especially example 6.

FIG. 6 (A) to (E) shows the results of immunohistochemical analyses using VIF, which are explained in detail in example 7; (D) and (E) clearly demonstrate the positive influence of VIF on the formation of new vessels.

FIG. 7 (A) and (B) show the results of the promotion of mitochondrial oxygen consumption rate as induced by VIF, as explained in more detail in example 8.

### DETAILED DESCRIPTION

According to the invention, the primary problem is solved by providing a vasoconstriction inhibitory factor (VIF) or a nucleic acid encoding it for use in the prevention and/or treatment of the consequences of a heart disease, preferably selected from the group consisting of coronary heart disease, myocarditis, myocardial infarction, myocardial ischemia and myocardial hypoxia.

Current therapies for heart diseases specialize in the long-term lowering of blood pressure to prevent recurrence of the same or a similar heart disease. Acute treatment strategies as well as tolerable and safe prevention strategies for patients at risk are urgently needed. There is also a high demand for maintenance therapies after a heart disease, for example to prevent worsening of the condition of heart tissue.

In the case of a myocardial infarction, for example, the blood supply to parts of the heart muscle is disturbed or interrupted. As a result, hypoxia occurs as a consequence of the lack of oxygen supply, which can lead to death of heart muscle cells and thus to inflammatory reactions in the heart tissue. However, such damage that has already occurred cannot be treated, or at least not satisfactorily and sustainably, by current forms of therapy. Moreover, even today, therapy by current forms of therapy is still not satisfactory. Patients often show no improvement in heart function, which is why often further, invasive therapies are necessary. Such therapies include among others cardiac resynchronization therapy (CRT), a biventricular pacemaker or an implantable cardioverter/defibrillator (ICD), which in turn carry a high risk of bleeding and infection.

Surprisingly, it was found that VIF, in addition to its influence on the renin-angiotensin system—and thus also on the regulation of blood pressure—has a protective effect in, for example, a myocardial infarction. Thus, a protective effect of VIF on heart muscles during a persistent circulatory disorder was surprisingly revealed and further characterized. The protective effect was shown by the fact that in pilot studies, the area affected by an infarction was significantly reduced by (pre-) treatment with VIF. Such an effect is not described in the state of the art for VIF and represents an enormous potential in the prevention of the consequences of a heart disease, especially in the context of a new approach that is specifically targeted to preventing or mitigating the consequences of a heart disease. By reducing the affected area, the resulting consequences will be mitigated and ultimately also therapy will be eased for the patients concerned (e.g. due to a lower drug dose or by weaker drugs with fewer side effects and thus higher patient compliance).

VIF was also found to improve the ejection fraction of the heart after a persistent circulatory disorder. The ejection fraction serves as a measure of heart function. Such an effect is also not described in the state of the art for VIF and represents a major advantage for a possible therapy, since previous therapies have failed to improve heart function. For example, a clinical benefit of the active substance RLX030 (Serelaxin) could not be achieved. In the Novartis RELAX-AHF-2 trial, Serelaxin did not reduce cardiovascular mortality in the first 180 days, nor did it reduce the worsening of cardiovascular disease in initially stabilized hospitalized patients in the first 5 days after the first episode of heart failure.

Studies in animal models have also shown that VIF surprisingly leads to both the formation of new blood vessels and increased mitochondrial oxygen consumption rates after an induced infarction. It is known that after an acute infarction, significant metabolic changes occur not only in the infarcted but also in the surviving non-infarcted segment (Mathes et al., 1974 Reduced contractility of the non-infarcted heart muscle after experimental infarction. In: Thauer R., Pleschka K. (Ed.) Das Arterielle System, Issue 40), leading to reduced contractility due to a reduced oxygen supply, among others. In the course of data collection in the context of the present invention, it could now surprisingly be shown that VIF does not only play a role in vasoconstriction. Rather, VIF also have specific properties that can play a major therapeutic role in both prevention and treatment of heart diseases. For example, it has been shown that VIF increases oxygen consumption rate of the mitochondrial respiratory chain in relevant cell types of the myocardium, thus contributing to increased myocardial contractility.

At the same time, the administration of VIF does not lead to an increased inflammatory reaction, which makes the peptide interesting for prophylactic as well as curative use. These specific effects of VIF have not been described to date and were not expected in view of the global mechanisms of action of VIF described above.

A vasoconstriction-inhibiting factor (VIF) is also preferred for use according to the invention, wherein the VIF contains an amino acid sequence according to SEQ ID NO: 1 (from homo sapiens) or an amino acid sequence having at least 90%, 91%, 92%, 93%, 94%, 95%, 96%, 97%, 98%, 99% sequence identity to the sequence according to SEQ ID NO: 1. An amino acid sequence according to SEQ ID NO: 1 or an amino acid sequence with at least 95% sequence identity to the sequence according to SEQ ID NO: 1 is preferred. Further preferred is an amino acid sequence in which an amino acid has been specifically substituted as compared to the sequence according to SEQ ID NO: 1, for example to investigate the effect/mechanism of action of VIF. SEQ ID NO: 1 describes the amino acid sequence of vasoconstriction-inhibiting factor (VIF) in its entire length.

In another embodiment, the VIF preferably contains an amino acid sequence according to SEQ ID NO: 2, SEQ ID NO: 3, SEQ ID NO: 4, SEQ ID NO: 5, SEQ ID NO: 6, SEQ ID NO: 7 or SEQ ID NO: 8 or an amino acid sequence having at least 90%, 91%, 92%, 93%, 94%, 95%, 96%, 97%, 98%, 99% sequence identity to the sequence according to SEQ ID NO: 2, SEQ ID NO: 3, SEQ ID NO: 4, SEQ ID NO: 5, SEQ ID NO: 6, SEQ ID NO: 7 or SEQ ID NO: 8. Preferred is an amino acid sequence according to SEQ ID NO: 2, SEQ ID NO: 3, SEQ ID NO: 4, SEQ ID NO: 5, SEQ ID NO: 6, SEQ ID NO: 7 or SEQ ID NO: 8 or an amino acid sequence with at least 95% sequence identity to the sequence according to SEQ ID NO: 2, SEQ ID NO: 3, SEQ ID NO: 4, SEQ ID NO: 5, SEQ ID NO: 6, SEQ ID NO: 7 or SEQ ID NO: 8. Further preferred is an amino acid sequence in which an amino acid has been specifically substituted as compared to the sequence according to SEQ ID NO: 2, SEQ ID NO: 3, SEQ ID NO: 4, SEQ ID NO: 5, SEQ ID NO: 6, SEQ ID NO: 7 or SEQ ID NO: 8, for example to investigate the effect/mechanism of action of VIF. SEQ ID NOs: 2 to 8 describe the amino acid sequences of individual peptides within the VIF (SEQ ID NO: 1).

The present invention further relates to a nucleic acid for use according to the invention, wherein the nucleic acid encodes an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8 or an amino acid sequence having at least 90%, 91%, 92%, 93%, 94%, 95%, 96%, 97%, 98%, 99% sequence identity to the sequence encoded by SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8. Preferred is an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8, or an amino acid sequence having at least 95% sequence identity to the sequence according to SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8. Further preferred is an amino acid sequence in which an amino acid has been specifically substituted as compared to the sequence according to SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8, for example to investigate the effect/mechanism of action of VIF.

Preferably, the present invention relates to a vasoconstriction-inhibiting factor (VIF) or a nucleic acid encoding it, for use according to the invention, wherein the prevention and/or the treatment involves a reduction of the area of heart tissue affected by the heart disease and/or a reduction of the limitation of cardiac output due to the heart disease.

In an embodiment, the present invention relates to a vasoconstriction-inhibiting factor (VIF) or a nucleic acid encoding it, for use according to the invention, wherein the prevention and/or the treatment of the consequences of the heart disease is achieved by increased vascularization as a result of VIF administration.

In a further embodiment, the present invention relates to a vasoconstriction-inhibiting factor (VIF) or a nucleic acid encoding it, for use according to the invention, wherein the prevention and/or the treatment of the consequences of the heart disease is achieved by an increased mitochondrial oxygen consumption rate as a result of VIF administration.

In yet another embodiment, the present invention relates to a vasoconstriction-inhibiting factor (VIF) or a nucleic acid encoding it, for use according to the invention, wherein the prevention and/or the treatment of the consequences of the heart disease is achieved by increased contractility of heart muscle cells as a result of the VIF administration. In particular, due to its influence on the respiratory chain of the mitochondria and hence on the oxygen turnover of a cell, the property of VIF to directly influence the metabolism of heart muscle cells suggests a dual therapeutic use of VIF: on the one hand, for prevention in patients at risk and/or predisposed to the development of coronary heart disease, and, in addition, for treatment after a myocardial infarction to strengthen the cells affected by the infarction.

In a further embodiment, the present invention relates to a vasoconstriction-inhibiting factor (VIF) or a nucleic acid encoding it, for use according to the invention, wherein the treatment of the consequences of the heart disease is achieved by increased monocyte infiltration into infarcted tissue as a result of VIF administration.

It is particularly advantageous for the prevention and/or the treatment of the consequences of the heart disease to keep the affected area of heart tissue as small as possible, especially after ischemia, in order to have to treat as little tissue as possible and to keep the expected loss of function as low as possible. This makes it possible to keep the required amount of medication and thus the potential side effects or long-term consequences low. The limitation of cardiac output is one of the most important and therefore most dangerous impairments, as a limited cardiac output can endanger the oxygen supply of the entire body. The smaller the impairment in cardiac output, the smaller the loss of heart function.

Further preferred is a vasoconstriction-inhibiting factor (VIF) or a nucleic acid encoding it, for use according to the invention, wherein the amino acid sequence, or the nucleic acid sequence encoding it, is produced by a fully synthetic method, by a biotechnological method or a combination of such methods, or wherein the production of the amino acid sequence, or the nucleic acid sequence encoding it, comprises a fully synthetic method, a biotechnological method or a combination of such methods.

In addition, a vasoconstriction-inhibiting factor (VIF) or a nucleic acid encoding it for use according to the invention is preferred, wherein the VIF is used, or applied, as a peptide, protein, optionally as a partial sequence of the VIF amino acid sequence and/or corresponding mimetics or as a nucleic acid and/or a mixture thereof, optionally together with at least one further pharmaceutically acceptable agent.

The dosage form of the VIF or its form when used, whether as a peptide, protein or nucleic acid, optionally as a partial sequence of the VIF amino acid sequence and/or corresponding mimetics and/or a mixture thereof, may have an influence on the spatial distribution of the VIF, its concentration in blood or its half-life. Preferably, excipients, as described herein, are used as pharmaceutically acceptable agents, in particular to influence the described properties of the VIF according to a corresponding interest. The described properties have particularly an influence on the available amount of VIF in the blood or heart tissue. A too fast or too slow degradation of VIF, or its half-life, plays a major role. It is preferable to avoid large fluctuations in VIF concentration in order to avoid possible over-or underdosage.

Preferably, the nucleic acid sequence or the amino acid sequence, optionally as a partial sequence of the VIF amino acid sequence and/or corresponding mimetics, contains at least one additional sequence, preferably wherein the at least one additional sequence has a stabilizing function, a marker function, an interaction function, a modulation function, or a localizing function. Preferably, the additional sequence, in the direction from the 5′ to the 3′ end of the nucleic acid, or in the direction from the C-terminal to the N-terminal end, is before or after the sequence of the VIF, or the sequence encoding the VIF, and not within this sequence. According to a preferred embodiment of the present invention, the at least one additional sequence does not negatively affect the activity of VIF. According to another embodiment of the present invention, the one or more of the at least one additional sequence(s) can influence the activity of VIF positively or negatively. According to another embodiment of the present invention, the at least one additional sequence does not affect the activity of VIF.

Additional functions such as a stabilizing function are of great advantage to counteract possible metabolization or degradation processes in the desired area of application (e.g. the human body, especially in the blood vessels and heart tissue), resulting in a longer lasting and more extensive availability in terms of area. Marker functions allow the tracing and thus marking of already treated tissues; the distribution in the application area can also be studied and analyzed by tracing. An interaction function can enable an interaction with previously selected, additional substances or tissues. Modulation functions allow an influence on e.g. the activity, which can for instance be bound to a certain residence time in the application area, wherein an activity before and/or after a previously selected time frame is no longer or only then possible. A localizing function represents, for example, a signal peptide, or a nucleic acid sequence or an amino acid sequence encoding it, which causes or initiates transport into a previously selected tissue. Such functions can be used to specifically influence the applicability, availability and activity of the VIF, or even of the nucleic acid sequence or of the amino acid sequence, in order to obtain an optimal result.

The present invention further concerns a pharmaceutical composition containing or consisting of
a) a vasoconstriction-inhibiting factor (VIF) as defined above,
b) optionally at least one excipient and/or additive, preferably wherein the at least one excipient and/or additive is selected from the group consisting of fillers, carriers, polymers, surfactants, disintegrants, binders, lubricants, sweeteners, flavorings, plasticizers, coating materials, cooling agents, recrystallization inhibitors, fluxes, defoamers, antioxidants, adsorbents, dyes, pH-modifying substances, preservatives, solvents, stabilizers, wetting agents, emulsifiers, salts for adjusting osmotic pressure and buffers, and
c) at least one further pharmaceutically active substance, wherein the at least one further pharmaceutically active substance is selected from statins, anticoagulants, beta blockers, ACE inhibitors, platelet aggregation inhibitors, Sartans, calcium antagonists, diuretics,
   preferably wherein said pharmaceutical composition is suitable for use in the prevention and/or treatment of consequences of a heart disease, preferably selected from the group consisting of coronary heart disease, myocarditis, myocardial infarction, myocardial ischemia and myocardial hypoxia, preferably wherein the prevention and/or the treatment involves a reduction of the area of the heart tissue affected by said heart disease and/or a reduction of the limitation of cardiac output due to said heart disease.

The protective effects of VIF on heart muscle cells described above can be advantageously used in a therapy for the prevention and/or treatment of the consequences of a heart disease. It is particularly advantageous to combine the therapy with other pharmaceutically active substances. For example, the blood pressure-lowering effects of the substances used so far can be combined with the protective effects of VIF. On the one hand, the occurrence of heart disease, as described above, can be prevented or delayed. In addition, the consequences of heart diseases as described above can be reduced or even prevented. Often high blood pressure-as an important cause of heart disease-is not detected in time before a heart disease occurs. Although the risk of the heart disease can then be reduced by lowering blood pressure, it is far from being eliminated. In this situation, however, an additional, preferably joint therapy with VIF, preferably within the context of a pharmaceutical composition according to the invention, can at least reduce or even prevent the consequences of the heart disease as described above, for example within the context of long-term therapy, but also within the context of short-term treatment.

In one embodiment, the pharmaceutical composition for use in treating the consequences of heart disease is one which is used in the context of post-infarction secondary prevention, wherein VIF is administered in combination with a statin and/or a beta-blocker and/or an anticoagulant and/or optionally an ACE inhibitor. Preferably, in maintenance therapy after myocardial infarction, the administration of an ACE inhibitor and/or an angiotensin II receptor blocker may be lowered when VIF is administered concomitantly.

In a further embodiment, a pharmaceutical composition is provided for use in the prevention of a heart disease, particularly in patients being at risk and high risk for developing coronary heart disease, or heart failure, wherein VIF is administered in combination with a statin and/or a beta-blocker and/or an anticoagulant and/or optionally an ACE inhibitor. In prophylaxis, VIF can be administered in particular together with a statin, or another lipid-lowering agent, to synergistically counteract atherosclerosis and plaque formation through the influence of the statin, as well as directly counteract the reduced activity of the cells of the heart tissue by VIF. In this way, the risk of a myocardial infarction, but also the risk of perioperative myocardial infarction as a complication, can be prevented.

The skilled person is aware that the choice of the at least one additional active ingredient administered in combination with VIF, as well as the concentration of VIF itself and of the at least one additional active ingredient and the treatment regimen and the dosage regimen, may depend on any underlying conditions, such as hypercholesterolemia.

Current therapies for the treatment of myocardial infarction are all incapable of alleviating acute necrosis in heart tissue and promoting regeneration of the infarcted tissue. Hypoxia leads to an induced death of cardiomyocytes as a result of an infarction. However, these direct damages are currently insufficiently treated by secondary therapies after a myocardial infarction.

Patients who have suffered a myocardial infarction are usually treated simultaneously with different groups of drugs following the myocardial infarction, usually with a combination of four or more preparations, often with a quadruple combination of an anticoagulant to inhibit blood clotting, such as ASA (acetylsalicylic acid) or clopidogrel, a statin to lower cholesterol, an ACE inhibitor or angiotensin II receptor blocker to lower blood pressure and a beta blocker to lower heart rate. The additional administration of VIF can reduce the use of antihypertensive drugs due to its vasodilative properties. At the same time, due to its influence on the formation of new blood vessels, as well as on the mitochondrial respiratory chain, and the recruitment of monocytes, VIF can also significantly contribute to the faster regeneration of infarcted and adjacent heart tissue and thus have an immediate positive influence in the treatment of acute coronary heart disease.

Likewise, cardiac risk patients can now be identified and adequately treated long before an infarction occurs. In one embodiment, VIF is therefore used preventively for cardio protective or vasculoprotective strategies, alone or in combination.

Therapy with VIF, preferably by way of a pharmaceutical composition according to the invention, can be performed in the context of an acute treatment, prevention and/or maintenance therapy.

Preferably, in a pharmaceutical composition according to the invention, components a) and c) are used in a pharmaceutically effective amount. This amount is typically a concentration of about 1 to 1,000 μg/kg body weight. In one embodiment, the dosage/dose of a VIF peptide according to the present invention is 1, 10, 30, 50, 100 or 250 μg/kg/day. In acute administration, the application of a higher concentration per day (in the range of about 250 μg/kg to 15,000 μg/kg, depending on the extent of the acute symptoms to be treated and further depending on patient-specific factors, the concentration may also be about 500 μg/kg to 10.000 μg/kg, about 750 μg/kg to 7,500 μg/kg, or about 500 μg/kg to 5,000 μg/kg) is preferred, in long-term or maintenance therapy a lower dose per day (<5,000 μg/kg, or even <1,000 μg/kg) can be administered. The dose may vary from one form of administration to another, as is known to the skilled person.

The pharmaceutical composition according to the invention can be applied systemically or locally. Preferred systemic applications are oral or parenteral such as intravenous, subcutaneous or endobronchial applications, applications per os, or an injection directly into the target tissue to be treated, preferably to induce a topical effect.

Preferably, the pharmaceutical composition according to the invention is in solid form, e.g. as powder, in liquid form, e.g. as an injection solution, or as an aerosol.

In addition, the present invention relates to a kit for non-therapeutic in vitro use containing the vasoconstriction inhibitory factor (VIF) or nucleic acid encoding it, as defined above.

Such a kit is particularly used to reveal the mechanisms of action and molecular mechanisms of VIF. In addition to the VIF or the nucleic acid encoding it, other contents may also be present. These include, for example, further compounds, substances or reagents that can be used for discovery work/research.

A kit can be provided in such a way that the contents are available in pre-measured quantities and/or concentrations so that they can be used directly or simply diluted to an applicable concentration. If other contents are present in addition to the VIF or the nucleic acid encoding it, these are preferably provided in a quantity or weight ratio to the VIF or the nucleic acid encoding it in which they are actually or approximately used.

The present invention is now further described by means of the attached non-limiting examples, the drawings and the sequence listing.

### EXAMPLES

### Example 1: Synthesis of the VIF

The VIF was automatically synthesized using the solid-phase method and standardized fluorenylmethyloxycarbonyl chloride chemistry via continuous-flow peptide synthesis.

### Example 2: Blood Pressure Lowering Effect of VIF

Male Wistar rats were used to investigate the in vivo effect of VIF. All animals had free access to standard rat food and tap water ad libitum. Mean arterial pressure was measured by a tail-cuff sphygmomanometer while conscious. Five determinations were made, the mean value serving as basal value. Subsequently, the animals received VIF intraperitoneally (1 mg per ml) and angiotensin II subcutaneously (0.4 mg per kg per day). The control group received only angiotensin II.

Subsequently, the blood pressure was measured for 30 to 45 minutes every 5 minutes via a microcatheter and determined using the software ADInstruments (Millar, Germany).

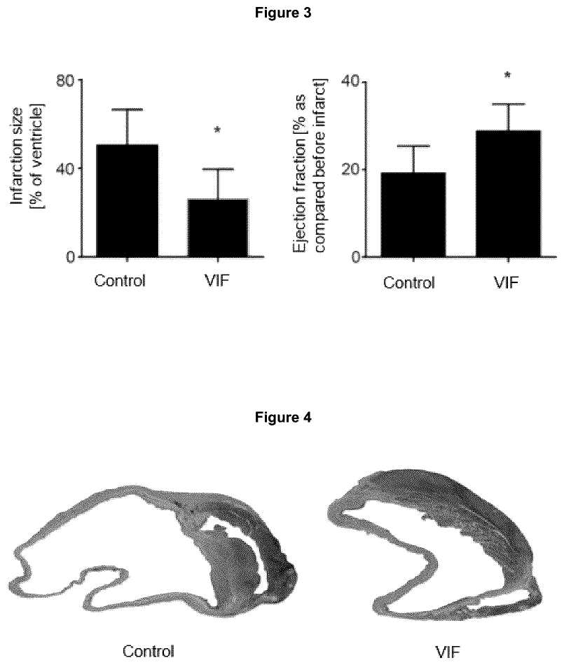

The results of the blood pressure measurements are shown in FIG. 2, wherein the groups with (▴) and without (▪) application of VIF are depicted.

### Example 3: Protective Effect of VIF on Heart Tissue

To study protective effects of VIF, mice were treated with VIF for 2 days before and 2 days after examination. Control animals received no treatment.

On the day of the examination a myocardial infarct was triggered. For this purpose, the mice were anesthetized by intraperitoneal injection of 100 mg/kg body weight ketamine and 10 mg/kg body weight xylazine and ventilated. The myocardial infarction was triggered by an occlusion of the LAD (left anterior descending artery).

Subsequently, the area of the affected tissue was determined by means of histological sections. For this purpose, the heart was removed, perfused with 1% Evans Blue, frozen for 2 h at −20° C. and then cut into 5 sections. The slices were incubated with preheated TTC solution for 10 min and fixed in formalin. Subsequently, images were taken and the infarcted area was calculated using DISKUS (Hilgard, Germany).

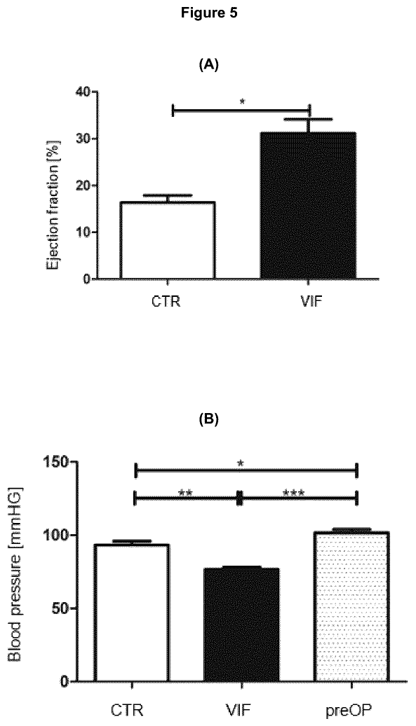

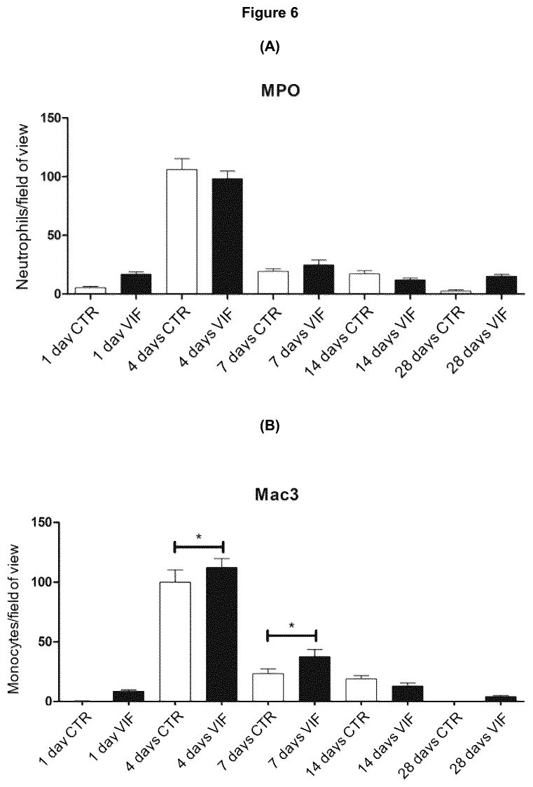

It was shown that in the animals treated with VIF the infarct size was only about half as large as in the control group. In animals treated with VIF, the tissue affected by the infarct occupied about 25% of the ventricle. In control animals, the affected tissue occupied about 50% of the ventricle. The results are shown in FIGS. 3 and 4.

The subsequent 2-day treatment with VIF also significantly improved the post-infarction ejection fraction. The ejection fraction of the control animals was almost 20% of the function in the healthy state, whereas the animals treated with VIF showed an ejection fraction of 30% of the function in the healthy state on average. The results are shown in FIG. 3.

### Example 4: Pharmaceutical Composition

The formulation of peptide-containing pharmaceutical compositions is determined by the solubility profile of the respective peptide of interest, its stability and the isoelectric point of the peptide as active ingredient. These characteristics also significantly determine the optimal pH value used in development and formulation. Especially the choice of the correct buffer system can be of great importance. Depending on the application route, peptide-containing pharmaceutical compositions are dissolved in a suitable physiologically compatible buffer/solvent system immediately prior to their application, if provided in powder or lyophilized form. Furthermore, the addition of stabilizers and preservatives is important, for example to prevent contamination of the peptide active ingredient. Stabilization can be particularly important for non-parenteral administration, if a certain half-life of the peptide in the patient must be reached in order for the peptide active ingredient to develop its activity over a given period of time. In addition, further excipients may be present. The use of excipients for delayed release may also be of importance in the context of the VIF peptides of the present invention, especially if they are used in long-term therapy. Suitable pharmaceutical compositions based on peptides are familiar to pharmacologists (see Pharmaceutical Formulation Development of Peptides and Proteins, edited by Lars Hovgaard, Sven Frokjaer, Marco van de Weert, Taylor & Francis, 2012).

For a pharmaceutical composition in accordance with the invention, the substances were provided in powder form, in solution or emulsion and mixed and, if necessary, dissolved one after the other. Different buffer systems were used under physiological conditions, depending on whether a full-length VIF peptide or one of the truncated variants was used (see Swain et al., Recent Patents on Biotechnology, 2013, 7). The mixture was then sterile filtered. Stability and functionality of the peptides was controlled by analytical in vitro experiments over time.

### Example 5: Animal Model Study on Myocardial Infarction

To further study the newly identified effects of VIF (according to SEQ ID NO: 1), and especially in vivo, 8 to 10 week old wild type male C57BL/6N mice (Charles River, Germany) were intubated under anesthesia (100 mg/kg ketamine, 10 mg/kg xylazine, i.p.) and analgesia (0.1 mg/kg buprenorphine). The mice were ventilated with positive pressure and oxygenation using a rodent respirator (Harvard Apparatus, Germany). A left-sided thoracotomy was then performed, and the MI (myocardial infarction) was induced by occlusion ligation of the left anterior descending artery (LAD) with 0/7 silk, as previously described in Curaj et al. (Minimally invasive surgical procedure of inducing myocardial infarction in mice. J Vis Exp. 2015: e52197). The rib, muscle and skin incisions were closed with separate sutures. Analgesia was continued for five days after the induced infarction using 0.1 mg/kg buprenorphine every eight hours. Thereafter, the hearts were removed at defined times (after 0, 1, 4, 7, 14, 21, 28 days) and prepared for further analysis.

VIF was dissolved at 6.7 μg/ml (1 mmol/l) in NaCl and loaded into 100 μl of Alzet-type 1002 osmotic pumps (0.25 μl/hour, Charles River, Cologne, Germany), resulting in a dose of 0.8 μg/kg per 24 hours. The Alzet pumps were implanted 24 hours before MI induction. The pumps for the controls were accordingly filled with NaCl only. All mice were kept under standardized conditions in the specially designed animal rooms of the University Hospital Aachen (Germany). All animal experiments and experimental protocols were approved by the local authorities in compliance with European and German animal welfare laws (84-02.04.2016.A315). All mice were included in the analysis, unless the animals had died during the experiment.

### Example 6: Echocardiography

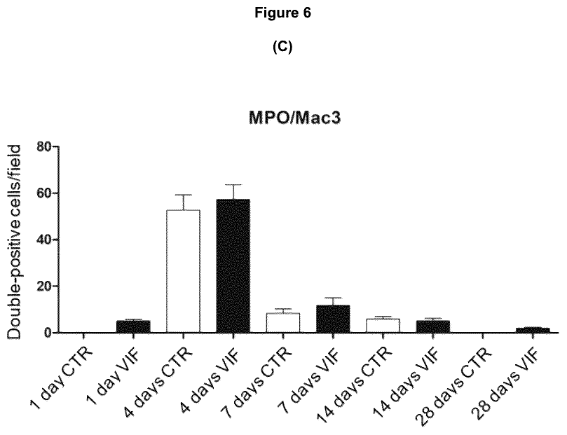

Two-dimensional as well as M-mode echocardiography measurements were performed with an ultrasound imager specifically designed for small animals (Vevo 770, FUJIFILM Visualsonics, Toronto, Canada). Both measurements were performed before and after myocardial infarct. For this purpose, mice were anesthetized with 1.5-2% isoflurane and placed on a warming pad in a supine position. The ejection fraction, cardiac output and heart rate were analyzed. The results are shown in FIG. 5.

The results indicate that VIF significantly increases the ejection fraction of the heart after treatment post-infarction (FIG. 5A), which was not expected to this extent based on the properties disclosed for VIF and may make a significant contribution to future treatments after myocardial infarction. In addition, there is a slight reduction in blood pressure (FIG. 5B).

### Example 7: Histology and Immunohistochemistry

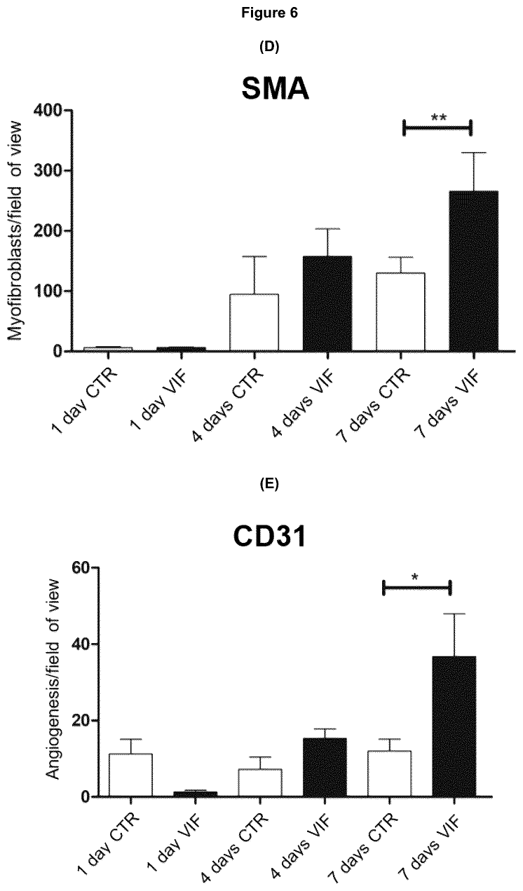

A Gömöri trichrome staining was performed to determine the infarct size. Subsequently, three slices per mouse from 3 to 5 different mice were analyzed using ImageJ. after one of anti-SMA antibody (smooth muscle actin, DAKO, FIG. 6D), anti-MAC3 antibody (BD Pharmingen, FIG. 6B), anti-MPO antibody (Neomarkers, FIG. 6A) and anti-CD31 antibody (Santa Cruz Biotechnology, FIG. 6E) staining, followed by staining with fluorescein isothiocyanate (FITC)—or a Cy3-conjugated secondary antibody (DAKO, Germany). Positively stained cells or double-positive stained cells (FIG. 6C for anti-MAC3/MPO) were counted in three different fields per slice and expressed as cells per field of view (200× magnification).

The results are shown in FIG. 6. On the one hand, these data clearly demonstrate (FIG. 6A and FIG. 6C) that VIF treatment does not lead to an increased infiltration of neutrophils visualized with anti-MPO compared to non-treated controls (CTR). This is an important indication of the therapeutic utility of VIF in that it does not induce acute cell-mediated inflammatory reactions. Following the invasion of neutrophils, invasion of a specific subpopulation of monocytes occurs in a second phase after a myocardial infarction. These Gr1-high expressing monocytes (Gr1+CCR2+CX3CR1 low) are characterized like human CD14^{high} CD16 monocytes, dominate the early phase of myocardial infarction and show phagocytic, proteolytic and inflammatory functions. Gr1-high expressing monocytes digest the infarcted heart tissue and remove cell debris from this area.

The number of monocytes (anti-MAC3 visualized) in VIF-treated animals behaved predominantly as in the control group. However, a slight but statistically significant increase was observed on day 7 in the single stain (FIG. 6B). This can be considered positive in that the infiltrating monocytes contribute to the removal of dead tissue and consequently to an improved regeneration, which can be positively influenced directly by the administration of VIF. This confirms that VIF can be used as a therapeutic agent after an acute myocardial infarction to promote and accelerate cell regeneration.

In principle, the results were therefore almost identical for VIF-treated animals and for the control group in the observed period of 28 days after induced infarction.

Surprising with regard to the function as vasoconstrictive factor originally disclosed for the VIF peptide was the newly discovered effect of vascularization that could be achieved after an induced infarction using VIF (FIG. 6D SMA and E CD31). FIG. 6D and E show the number of SMA and CD31 positive cells in the field of view, respectively, with CD31 used as a marker for endothelial cells and SMA as a marker for smooth muscle cells. For both markers, a statistically significant level of increased myofibroblast count and angiogenesis, both indicative of vascularization, was observed in the VIF treated groups on day 7.

Thus, surprisingly, in the critical phase after an infarction, which can damage large parts of the heart tissue, VIF administration leads to accelerated neovascularization as the basis for healing of the tissue damaged by the infarction. This makes VIF an interesting candidate in therapy after an acute myocardial infarction to specifically promote the formation of new blood vessels and thus minimize the damage caused.

### Example 8: Energy Catabolism Measurements

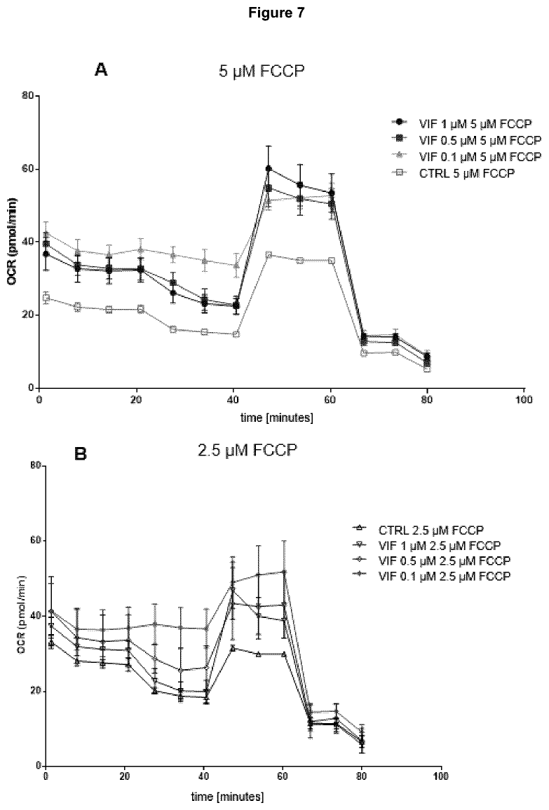

To further investigate the post-infarction role of VIF in the animal model described above, a measurement of tissue O₂ consumption (OCR for oxygen consumption rate) of VIF-treated and non-treated animals after the induced infarction was performed (Agilent Seahorse XF Cell Mito Stress Test Kit; Seahorse Bioanalyzer, Seahorse Bioscience). Thereby, the mitochondrial function of cells is determined. FCCP (carbonyl cyanide-4 (trifluoromethoxy) phenylhydrazone) is used as modulator. The experiments were performed using 5 μM FCCP (FIG. 7A) as well as 2.5 μM FCCP (FIG. 7B) on HL-1 cells (heart muscle cell line). The FCCP administration leads to an induced collapse of the proton gradient and thus interrupts the mitochondrial membrane potential. FCCP-stimulated OCR can therefore be used to determine the delta between maximum and basal activity. This delta, in turn, is a measure of how well a cell is able to respond to increased energy requirements (for example, after stress).

As illustrated in FIGS. 7A and B, VIF-treated cells (VIF each titrated from 0.1 to 1 μM) were always able to achieve significantly higher OCR values than the untreated control cells (CTRL).

This means that VIF can increase the maximum myocardial oxygen turnover, thereby increasing the contractility of heart muscle cells, among others. This can make a decisive contribution to both the prophylaxis and therapy of coronary heart diseases, as the relevant OCR values can be specifically influenced by modulating the respiratory chain reaction. These results prove that VIF can unexpectedly influence other metabolic processes as a specific modulator in addition to its role as a vasoconstrictive factor. Currently, these effects are being further investigated for the other VIF variants (according to SEQ ID NOs: 2 to 8) by in vitro and in vivo analyses.

### Example 9: Statistical Analysis

The statistical data shown in the figures represent the mean value ±SEM (standard error of the mean value). The statistical analysis was performed using Prism 7 software (GraphPad). The means of two groups were compared with the unpaired student t-test, using the Welch correction for significant variance. More than two groups were analyzed using a single factor ANOVA analysis of variance followed by a Newman-Keuls post hoc test, or a two-factor ANOVA analysis of variance followed by a Bonferroni multiple comparison test, in the case of more than two variable parameters as indicated. P-values of <0.05 were considered significant.

1.-15. (canceled)

16. A method for treating a patient, comprising the steps(s) of:
providing a patient at risk for cardiac tissue damage to reduce cardiac tissue damage by treating with at least one vasoconstriction-inhibiting factor (VIF) polypeptide or a nucleic acid that encodes said one at least VIF polypeptide, wherein the patient is at risk of cardiac tissue damage is due to one or more conditions selected from the group consisting of: coronary heart disease, myocarditis, myocardial infarction, myocardial ischemia and myocardial hypoxia.

17. The method according to claim 16, wherein the VIF polypeptide comprises one or more amino sequences selected from the group consisting of: SEQ ID NO: 1 or an amino acid sequence having at least 90%, 91%, 92%, 93%, 94%, 95%, 96%, 97%, 98%, 99% sequence identity to the sequence according to SEQ ID NO: 1.

18. The method according to claim 16, wherein the VIF polypeptide comprises one or more amino sequences selected from the group consisting of: SEQ ID NO:2, SEQ ID NO:3, SEQ ID NO:4, SEQ ID NO:5, SEQ ID NO:6, SEQ ID NO:7 or SEQ ID NO: 8, an amino acid sequence having at least 90%, 91%, 92%, 93%, 94%, 95%, 96%, 97%, 98%, or 99% sequence identity to the sequences according to SEQ ID NO: 2, SEQ ID NO: 3, SEQ ID NO: 4, SEQ ID NO: 5, SEQ ID NO: 6, SEQ ID NO: 7 or SEQ ID NO: 8.

19. The method according to claim 16, wherein the nucleic acid that encodes said one at least VIF polypeptide is at least one nucleic acid that encodes at least one polypeptide selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8, an amino acid sequence having at least 90%, 91%, 92%, 93%, 94%, 95%, 96%, 97%, 98%, 99% sequence identity to the sequence according to SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8.

20. The method according to claim 16, wherein VIF exhibits one or more of the following effects on cardiac tissue selected from the group of effects consisting of: a reduction of the area of heart tissue affected by heart disease, a reduction of the limitation of cardiac output due to heart disease, an increase in vascularization of damaged cardia tissue, an increase in mitochondrial oxygen consumption rate, an increase in contractility of heart muscle cells, and an increase in monocyte infiltration into infarcted cardiac tissue.

21. The method according to claim 16, wherein the VIF polypeptide or the VIF nucleic acid that encodes the VIF polypeptide is selected from the at least one of the following production methods: a fully synthetic method, a biotechnological method, and a combination of synthetic and biotechnological methods.

22. The method according to claim 16, further including the step of:
treating the patient with one or more additional therapeutic compounds.

23. The method according to claim 16, wherein the VIF polypeptide or the VIF nucleic acid that encodes the VIF polypeptide is modified to include at least one of the modifiers selected from the group consisting of: a stabilizer, a marker, a localizer, and a modulator.
